(19) United States
(12) Patent Application Publication
(10) Pub. No.: US 2015/0031971 A1
(43) Pub. Date: Jan. 29, 2015

(54) METHODS AND SYSTEMS FOR USING AN ESTIMATE SIGNAL TO IMPROVE SIGNAL RESOLUTION IN A PHYSIOLOGICAL MONITOR

(71) Applicant: Covidien LP, Mansfield, MA (US)

(72) Inventors: Daniel Lisogurski, Boulder, CO (US); Christopher Meehan, Golden, CO (US)

(73) Assignee: Covidien LP, Mansfield, MA (US)

(21) Appl. No.: 13/952,534

(22) Filed: Jul. 26, 2013

(51) Int. Cl. A61B 5/1455; A61B 5/021; A61B 5/08; A61B 5/024

(52) U.S. Cl. CPC A61B 5/14552; A61B 5/02416; A61B 5/021; A61B 5/1455; A61B 5/08
USPC 600/324; 600/479; 600/480

(57) ABSTRACT

A physiological monitoring system may receive a sensor signal from a physiological sensor. The system may generate an estimate of the sensor signal based on, for example, prior received signals. The estimate signal may be subtracted from the sensor signal using a transimpedance amplifier to generate a difference signal. A gain and/or offset may be applied to the difference signal by the amplifier. The amplified difference signal may be digitized and combined with the estimate signal to generate a high resolution digital representation of the sensor signal. Physiological information such as blood oxygen saturation, pulse rate, respiration rate, respiration effort, blood pressure, hemoglobin concentration, any other suitable physiological parameters, or any combination thereof, may be determined using the digitized sensor signal. In some embodiments, the use of the signal estimate and the amplified difference signal in processing physiological parameters may provide high resolution without high power and/or processing requirements.

The present disclosure relates to operating a physiological monitor, and more particularly relates to using an estimate of a sensor signal to generate a difference signal for use in a pulse oximeter or other medical device.

### SUMMARY

The present disclosure is directed towards the processing of signals in a physiological monitoring system such as a medical device. Methods and systems are provided for using an estimate signal in a physiological monitor. The estimate signal may include any suitable non-physiological and constant physiological contributions to a sensor signal. The estimate signal may be generated based on prior received signals. The estimate signal may be subtracted from a sensor signal to generate a difference signal, where the difference signal is primarily composed of varying physiological information. The difference signal may be amplified, digitized, and combined with the estimate signal to construct a digital representation of the physiological signal. By amplifying the difference signal rather than the sensor signal, a relatively high gain may be utilized, and a relatively high resolution digital representation of the physiological information may be achieved. The representation of the physiological signal may be used to determine physiological parameters such as blood oxygen saturation, pulse rate, and respiration rate.

In an example, a sensor signal from a photodetector of a pulse oximeter and an estimate signal generated by a digital-to-analog converter may be provided to the inverting and non-inverting inputs, respectively, of a transimpedance amplifier. The amplifier may output a difference signal that is digitized using an analog-to-digital converter. The amplifier may apply a gain and/or offset such that the utilization of the dynamic range of the analog-to-digital converter is optimized. In some embodiments, the use of an estimate signal may result in a relatively high resolution representation of the modulations in the physiological signal that correspond to physiological parameters, while reducing the processing and/or power requirements.

In some embodiments, a method for processing a sensor signal in a physiological monitor is provided. The method comprises receiving, using processing equipment, an analog sensor signal corresponding to an intensity of detected light attenuated by a subject, where the analog sensor signal comprises at least a first segment corresponding to an ambient light interval and a second segment correspond to an emitted light interval, and where the sensor signal comprises physiological information. The method further comprises generating, using the processing equipment, an analog estimate signal, where the estimate comprises an estimate of at least a portion of the first and second segments. The method further comprises generating, using the processing equipment, an analog difference signal based at least in part on the analog sensor signal and the analog estimate signal. The method further comprises determining, using the processing equipment, a physiological parameter of the subject based at least in part on the analog difference signal.

In some embodiments, a system for processing a sensor signal in a physiological monitor is provided. The system comprises processing equipment configured to perform operations. The operations comprise receiving an analog sensor signal corresponding to an intensity of detected light attenuated by a subject, where the analog sensor signal comprises at least a first segment corresponding to an ambient light interval and a second segment correspond to an emitted light interval, and where the sensor signal comprises physiological information. The operations further comprise generating an analog estimate signal, where the estimate comprises an estimate of at least a portion of the first and second segments. The operations further comprise generating an analog difference signal based at least in part on the analog sensor signal and the analog estimate signal. The operations further comprise determining a physiological parameter of the subject based at least in part on the analog difference signal.

In some embodiments, a method for determining a physiological parameter of a subject is provided. The method comprises receiving a signal representing intensity of light attenuated by a subject. The method further comprises generating an estimate signal based on an idealized light intensity signal. The method further comprises generating a difference signal representing a difference between the received signal and the estimate signal. The method further comprises amplifying the difference signal. The method further comprises determining a physiological parameter of the subject based at least in part on the amplified difference signal.

### BRIEF DESCRIPTION OF THE FIGURES

The above and other features of the present disclosure, its nature and various advantages will be more apparent upon consideration of the following detailed description, taken in conjunction with the accompanying drawings in which:

FIG. 1 is a block diagram of an illustrative physiological monitoring system in accordance with some embodiments of the present disclosure;

FIG. 2A shows an illustrative plot of a light drive signal in accordance with some embodiments of the present disclosure;

FIG. 2B shows an illustrative plot of a detector signal that may be generated by a sensor in accordance with some embodiments of the present disclosure;

FIG. 3 is a perspective view of an illustrative physiological monitoring system in accordance with some embodiments of the present disclosure;

FIG. 4 is a block diagram of an illustrative system including an estimate signal in accordance with some embodiments of the present disclosure;

FIG. 5 shows illustrative signal plots in accordance with some embodiments of the present disclosure; and

FIG. 6 shows an illustrative flow diagram including steps for determining a physiological parameter using an estimate of a sensor signal in accordance with some embodiments of the present disclosure.

### DETAILED DESCRIPTION OF THE FIGURES

The present disclosure is directed towards processing of signals in a physiological monitoring system such as a medical device. A physiological monitoring system may receive a signal from a physiological sensor attached to a patient. The system may generate an estimate of the received signal based on prior signals. The estimate may make use of the repeating signal levels that occur in a sensor signal, for example those corresponding to a light drive modulation. In some embodiments, the estimate of the signal may include non-physiological and constant physiological contributions to the sensor signal. The system may generate a difference signal by subtracting the estimate signal from the sensor signal, and amplify the remaining difference signal. The amplified difference signal may be input to an analog-to-digital converter (ADC) and combined digitally with the estimate signal to construct a digital representation of the sensor signal. In some embodiments, a relatively higher gain may be applied to the difference signal than would have otherwise been applied to the sensor signal. This increased amplification may result in relatively increased resolution of the digitized sensor signal, and may additionally or alternatively reduce power requirements of the system. In some embodiments, using an estimate signal and generating a difference signal may allow for lower voltage supply rails, which may result in reduced integrated circuit board area allocated to power supply, lower power consumption, lower cost, other benefits, and combinations thereof.

In an example using an optical pulse oximeter, the estimate signal may include signal levels corresponding to light sources turning on and off, as well as ambient light sources and electronic noise. Modulations remaining in the difference signal may then correspond primarily to physiological parameters such as blood volume modulation, which may be less than 1% of the modulation in the received sensor signal. In some embodiments, physiological modulations, such as those corresponding to cardiac pulse and/or respiration, may also be included in the estimate signal, in which case the difference signal includes only unexpected changes in the physiological information. The characteristics of the sensor signal, for example, peak height, shape, and periodicity, may change relatively slowly, allowing the use of prior signal information in generating the estimate. That is to say, the sensor signal may include relatively regular, repeating features that are substantially predictable over a short period of time. The repeating features may be used in generating an estimate signal.

In some embodiments, the subtraction of the estimate signal from the sensor signal may be performed using an amplifier such as a transimpedance amplifier, a differential amplifier, any other suitable amplifier, or any combination thereof. In an example, a transimpedance amplifier is used in a pulse oximeter system, where the transimpedance amplifier outputs a voltage based on the difference between a sensor current received from a photodetector, and an estimated current received from a digital-to-analog converter (DAC). In an example, an amplifier may receive the sensor signal at a non-inverting input and the estimate signal at an inverting input. The output of the amplifier may be the result of subtracting the estimate signal from the sensor signal. In some embodiments, power supply rails may supply lower voltage, and thus reduce power consumption, because the power supply to the amplifier need only account for the dynamic range of the difference signal, and not for the dynamic range of the sensor signal, which may encompass, for example, on and off periods of a light source such as an LED.

The amplifier may include any suitable gain, including gains equal to and less than 1. In an example, a transimpedance amplifier may be used with a differential input, a single output, and a gain of 230,000. A relatively high gain may be applied to the difference signal because it includes a relatively small portion of the sensor signal amplitude. In some embodiments, the gain may initially be set to 1 and may be adjusted such that the utilization of the dynamic range of an analog-to-digital converter (ADC) receiving the amplified difference signal is optimized. In some embodiments, an offset may be applied to the difference signal in order to align the difference signal with the dynamic range of an ADC input.

The foregoing techniques may be implemented in an oximeter. An oximeter is a medical device that may determine the oxygen saturation of an analyzed tissue. One common type of oximeter is a pulse oximeter, which may non-invasively measure the oxygen saturation of a patient's blood (as opposed to measuring oxygen saturation invasively by analyzing a blood sample taken from the patient). Pulse oximeters may be included in patient monitoring systems that measure and display various blood flow characteristics including, but not limited to, the blood oxygen saturation (e.g., arterial, venous, or both). Such patient monitoring systems may also measure and display additional or alternative physiological parameters such as pulse rate, respiration rate, respiration effort, blood pressure, hemoglobin concentration (e.g., oxygenated, deoxygenated, and/or total), any other suitable physiological parameters, or any combination thereof. Exemplary embodiments of determining respiration rate are disclosed in Addison et al. U.S. Patent Publication No. 2011/0071406, published Mar. 24, 2011, which is hereby incorporated by reference herein in its entirety. Exemplary embodiments of determining respiration effort are disclosed in Addison et al. U.S. Patent Publication No. 2011/0004081, published Jan. 6, 2011, which is hereby incorporated by reference herein in its entirety. Exemplary embodiments of determining blood pressure are disclosed in Addison et al. U.S. Patent Publication No. 2011/0028854, published Feb. 3, 2011, which is hereby incorporated by reference herein in its entirety.

Pulse oximetry may be implemented using a photoplethysmograph. Pulse oximeters and other photoplethysmograph devices may also be used to determine other physiological parameter and information as disclosed in: J. Allen, “Photoplethysmography and its application in clinical physiological measurement,” Physiol. Meas., vol. 28, pp. R1-R39, March 2007; W. B. Murray and P. A. Foster, “The peripheral pulse wave: information overlooked,” J. Clin. Monit., vol. 12, pp. 365-377, September 1996; and K. H. Shelley, “Photoplethysmography: beyond the calculation of arterial oxygen saturation and heart rate,” Anesth. Analg., vol. 105, pp. S31-S36, December 2007; all of which are incorporated by reference herein in their entireties.

An oximeter may include a light sensor that is placed at a site on a patient, typically a fingertip, toe, forehead or earlobe, or in the case of a neonate, across a foot or hand. The oximeter may use a light source to pass light through blood perfused tissue and photoelectrically sense the absorption of the light in the tissue. Additional suitable sensor locations include, without limitation, the neck to monitor carotid artery pulsatile flow, the wrist to monitor radial artery pulsatile flow, the inside of a patient's thigh to monitor femoral artery pulsatile flow, the ankle to monitor tibial artery pulsatile flow, around or in front of the ear, and locations with strong pulsatile arterial flow. Suitable sensors for these locations may include sensors that detect reflected light.

The oximeter may measure the intensity of light that is received at the light sensor as a function of time. The oximeter may also include sensors at multiple locations. A signal representing light intensity versus time or a mathematical manipulation of this signal (e.g., a scaled version thereof, a log taken thereof, a scaled version of a log taken thereof, an inverted signal, etc.) may be referred to as the photoplethysmograph (PPG) signal. In addition, the term “PPG signal,” as used herein, may also refer to an absorption signal (i.e., representing the amount of light absorbed by the tissue) or any suitable mathematical manipulation thereof. The light intensity or the amount of light absorbed may then be used to calculate any of a number of physiological parameters, including an amount of a blood constituent (e.g., oxyhemoglobin) being measured as well as a pulse rate and when each individual pulse occurs.

In some embodiments, the photonic signal interacting with the tissue is of one or more wavelengths that are attenuated by the blood in an amount representative of the blood constituent concentration. Red and infrared (IR) wavelengths may be used because it has been observed that highly oxygenated blood will absorb relatively less red light and more IR light than blood with a lower oxygen saturation. By comparing the intensities of two wavelengths at different points in the pulse cycle, it is possible to estimate the blood oxygen saturation of hemoglobin in arterial blood.

The system may process data to determine physiological parameters using techniques well known in the art. For example, the system may determine blood oxygen saturation using two wavelengths of light and a ratio-of-ratios calculation. The system also may identify pulses and determine pulse amplitude, respiration, blood pressure, other suitable parameters, or any combination thereof, using any suitable calculation techniques. In some embodiments, the system may use information from external sources (e.g., tabulated data, secondary sensor devices) to determine physiological parameters.

In some embodiments, a light drive modulation may be used. For example, a first light source may be turned on for a first drive pulse, followed by an off period, followed by a second light source for a second drive pulse, followed by an off period. The first and second drive pulses may be used to determine physiological parameters. The off periods may be used to detect ambient signal levels, reduce overlap of the light drive pulses, allow time for light sources to stabilize, allow time for detected light signals to stabilize or settle, reduce heating effects, reduce power consumption, for any other suitable reason, or any combination thereof.

It will be understood that the estimate signal techniques described herein are not limited to pulse oximeters and may be applied to any suitable medical and non-medical devices. For example, the system may include probes for parameters such as regional saturation (rSO₂), respiration rate, respiration effort, continuous non-invasive blood pressure, oxygen saturation pattern detection, fluid responsiveness, cardiac output, any other suitable clinical parameter, or any combination thereof, and the system may generate estimate signals and difference signals in order to calculate such parameter(s).

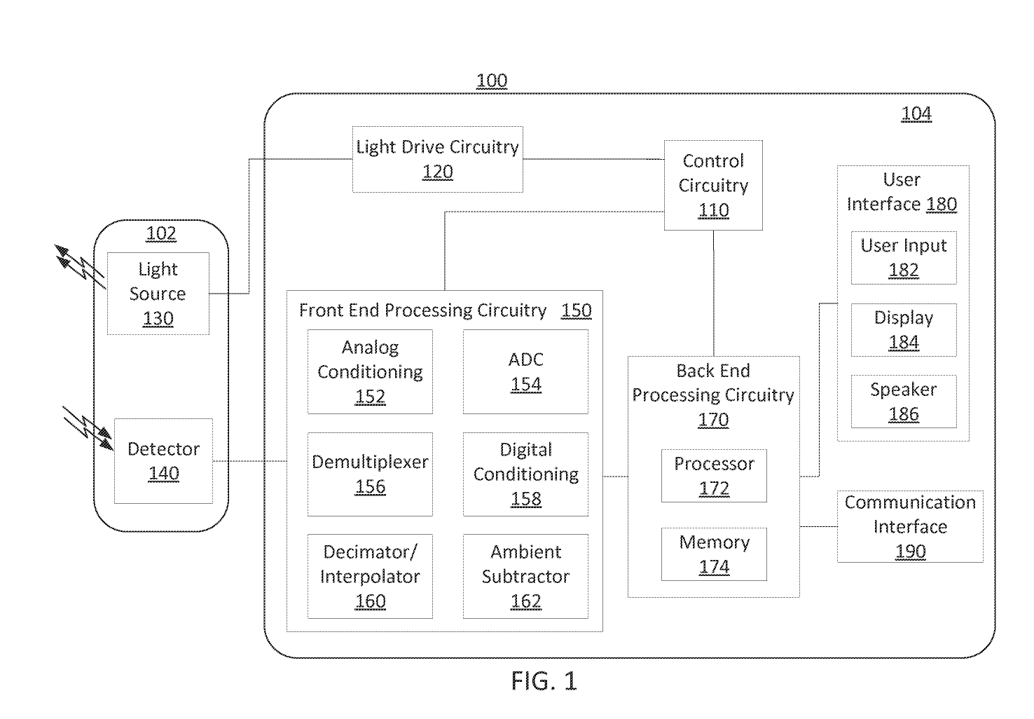

The following description and accompanying FIGS. 1-6 provide additional details and features of some embodiments of the present disclosure.

FIG. 1 is a block diagram of an illustrative physiological monitoring system 100 in accordance with some embodiments of the present disclosure. System 100 may include a sensor 102 and a monitor 104 for generating and processing sensor signals that include physiological information of a subject. In some embodiments, sensor 102 and monitor 104 may be part of an oximeter.

Sensor 102 of physiological monitoring system 100 may include light source 130 and detector 140. Light source 130 may be configured to emit photonic signals having one or more wavelengths of light (e.g. red and IR) into a subject's tissue. For example, light source 130 may include a red light emitting light source and an IR light emitting light source, e.g. red and IR light emitting diodes (LEDs), for emitting light into the tissue of a subject to generate sensor signals that include physiological information. In one embodiment, the red wavelength may be between about 600 nm and about 700 nm, and the IR wavelength may be between about 800 nm and about 1000 nm. It will be understood that light source 130 may include any number of light sources with any suitable characteristics. In embodiments where an array of sensors is used in place of single sensor 102, each sensor may be configured to emit a single wavelength. For example, a first sensor may emit only a red light while a second may emit only an IR light.

It will be understood that, as used herein, the term “light” may refer to energy produced by radiative sources and may include one or more of ultrasound, radio, microwave, millimeter wave, infrared, visible, ultraviolet, gamma ray or X-ray electromagnetic radiation. As used herein, light may also include any wavelength within the radio, microwave, infrared, visible, ultraviolet, or X-ray spectra, and that any suitable wavelength of electromagnetic radiation may be appropriate for use with the present techniques. Detector 140 may be chosen to be specifically sensitive to the chosen targeted energy spectrum of light source 130.

In some embodiments, detector 140 may be configured to detect the intensity of light at the red and IR wavelengths. In some embodiments, an array of sensors may be used and each sensor in the array may be configured to detect an intensity of a single wavelength. In operation, light may enter detector 140 after passing through the subject's tissue. Detector 140 may convert the intensity of the received light into an electrical signal. The light intensity may be directly related to the absorbance and/or reflectance of light in the tissue. That is, when more light at a certain wavelength is absorbed or reflected, less light of that wavelength is received from the tissue by detector 140. After converting the received light to an electrical signal, detector 140 may send the detection signal to monitor 104, where the detection signal may be processed and physiological parameters may be determined (e.g., based on the absorption of the red and IR wavelengths in the subject's tissue). In some embodiments, the detection signal may be preprocessed by sensor 102 before being transmitted to monitor 104.

In the embodiment shown, monitor 104 includes control circuitry 110, light drive circuitry 120, front end processing circuitry 150, back end processing circuitry 170, user interface 180, and communication interface 190. Monitor 104 may be communicatively coupled to sensor 102.

Control circuitry 110 may be coupled to light drive circuitry 120, front end processing circuitry 150, and back end processing circuitry 170, and may be configured to control the operation of these components. In some embodiments, control circuitry 110 may be configured to provide timing control signals to coordinate their operation. For example, light drive circuitry 120 may generate a light drive signal, which may be used to turn on and off the light source 130, based on the timing control signals. The front end processing circuitry 150 may use the timing control signals to operate synchronously with light drive circuitry 120. For example, front end processing circuitry 150 may synchronize the operation of an analog-to-digital converter and a demultiplexer with the light drive signal based on the timing control signals. In addition, the back end processing circuitry 170 may use the timing control signals to coordinate its operation with front end processing circuitry 150.

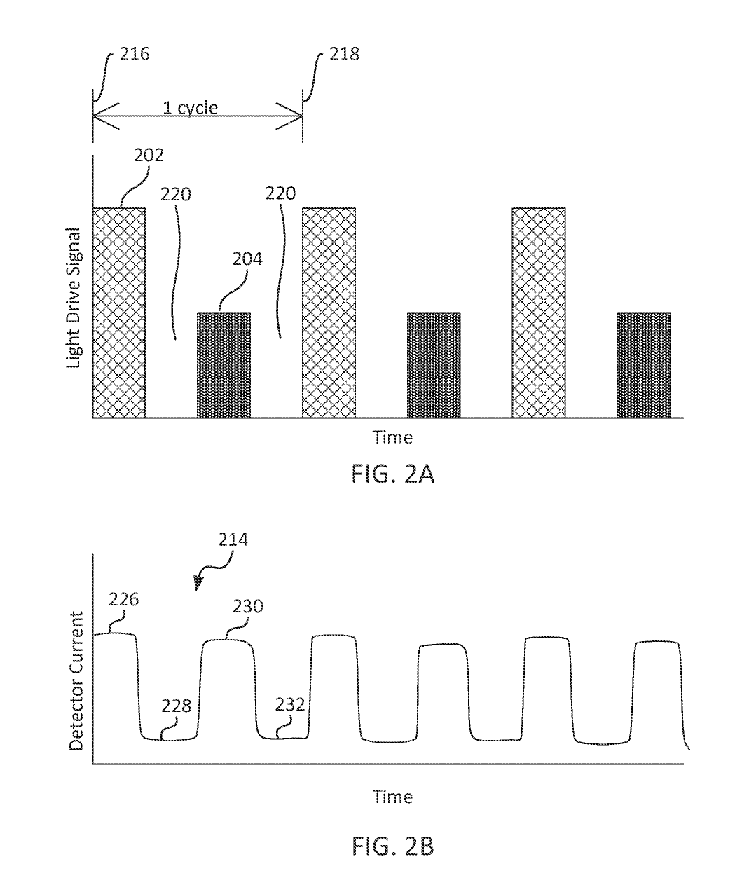

Light drive circuitry 120, as discussed above, may be configured to generate a light drive signal that is provided to light source 130 of sensor 102. The light drive signal may, for example, control the intensity of light source 130 and the timing of when light source 130 is turned on and off. When light source 130 is configured to emit two or more wavelengths of light, the light drive signal may be configured to control the operation of each wavelength of light. The light drive signal may comprise a single signal or may comprise multiple signals (e.g., one signal for each wavelength of light). An illustrative light drive signal is shown in FIG. 2A.

In some embodiments, control circuitry 110 and light drive circuitry 120 may generate light drive parameters based on a metric. For example, back end processing 170 may receive information about received light signals, determine light drive parameters based on that information, and send corresponding information to control circuitry 110.

FIG. 2A shows an illustrative plot of a light drive signal including red light drive pulse 202 and IR light drive pulse 204 in accordance with some embodiments of the present disclosure. Light drive pulses 202 and 204 are illustrated as square waves. These pulses may include shaped waveforms rather than a square wave. The shape of the pulses may be generated by a digital signal generator, digital filters, analog filters, any other suitable equipment, or any combination thereof. For example, light drive pulses 202 and 204 may be generated by light drive circuitry 120 under the control of control circuitry 110. As used herein, drive pulses may refer to the high and low states of a shaped pulse, switching power or other components on and off, high and low output states, high and low values within a continuous modulation, other suitable relatively distinct states, or any combination thereof. The light drive signal may be provided to light source 130, including red light drive pulse 202 and IR light drive pulse 204 to drive red and IR light emitters, respectively, within light source 130. Red light drive pulse 202 may have a higher amplitude than IR light drive 204 since red LEDs may be less efficient than IR LEDs at converting electrical energy into light energy. In some embodiments, the output levels may be equal, may be adjusted for nonlinearity of emitters, may be modulated in any other suitable technique, or any combination thereof. Additionally, red light may be absorbed and scattered more than IR light when passing through perfused tissue.

When the red and IR light sources are driven in this manner they emit pulses of light at their respective wavelengths into the tissue of a subject in order generate sensor signals that include physiological information that physiological monitoring system 100 may process to calculate physiological parameters. It will be understood that the light drive amplitudes of FIG. 2A are merely exemplary and that any suitable amplitudes or combination of amplitudes may be used, and may be based on the light sources, the subject tissue, the determined physiological parameter, modulation techniques, power sources, any other suitable criteria, or any combination thereof.

The light drive signal of FIG. 2A may also include “off” periods 220 between the red and IR light drive pulse. “Off” periods 220 are periods during which no drive current may be applied to light source 130. “Off” periods 220 may be provided, for example, to prevent overlap of the emitted light, since light source 130 may require time to turn completely on and completely off. The period from time 216 to time 218 may be referred to as a drive cycle, which includes four segments: a red light drive pulse 202, followed by an “off” period 220, followed by an IR light drive pulse 204, and followed by an “off” period 220. After time 218, the drive cycle may be repeated (e.g., as long as a light drive signal is provided to light source 130). It will be understood that the starting point of the drive cycle is merely illustrative and that the drive cycle can start at any location within FIG. 2A, provided the cycle spans two drive pulses and two “off” periods. Thus, each red light drive pulse 202 and each IR light drive pulse 204 may be understood to be surrounded by two “off” periods 220. “Off” periods may also be referred to as dark periods, in that the emitters are dark or returning to dark during that period. It will be understood that the particular square pulses illustrated in FIG. 2A are merely exemplary and that any suitable light drive scheme is possible. For example, light drive schemes may include shaped pulses, sinusoidal modulations, time division multiplexing other than as shown, frequency division multiplexing, phase division multiplexing, any other suitable light drive scheme, or any combination thereof.

Referring back to FIG. 1, front end processing circuitry 150 may receive a detection signal from detector 140 and provide one or more processed signals to back end processing circuitry 170. The term “detection signal,” as used herein, may refer to any of the signals generated within front end processing circuitry 150 as it processes the output signal of detector 140. Front end processing circuitry 150 may perform various analog and digital processing of the detector signal. One suitable detector signal that may be received by front end processing circuitry 150 is shown in FIG. 2B.

FIG. 2B shows an illustrative plot of detector current waveform 214 that may be generated by a sensor in accordance with some embodiments of the present disclosure. The peaks of detector current waveform 214 may represent current signals provided by a detector, such as detector 140 of FIG. 1, when light is being emitted from a light source. The amplitude of detector current waveform 214 may be proportional to the light incident upon the detector. The peaks of detector current waveform 214 may be synchronous with drive pulses driving one or more emitters of a light source, such as light source 130 of FIG. 1. For example, detector current peak 226 may be generated in response to a light source being driven by red light drive pulse 202 of FIG. 2A, and peak 230 may be generated in response to a light source being driven by IR light drive pulse 204. Valley 228 of detector current waveform 214 may be synchronous with periods of time during which no light is being emitted by the light source, or the light source is returning to dark, such as “off” period 220. While no light is being emitted by a light source during the valleys, detector current waveform 214 may not fall all of the way to zero.

It will be understood that detector current waveform 214 may be an at least partially idealized representation of a detector signal, assuming perfect light signal generation, transmission, and detection. It will be understood that an actual detector current will include amplitude fluctuations, frequency deviations, droop, overshoot, undershoot, rise time deviations, fall time deviations, other deviations from the ideal, or any combination thereof. It will be understood that the system may shape the drive pulses shown in FIG. 2A in order to make the detector current as similar as possible to idealized detector current waveform 214.

Referring back to FIG. 1, front end processing circuitry 150, which may receive a detection signal, such as detector current waveform 214, may include analog conditioning 152, analog-to-digital converter (ADC) 154, demultiplexer 156, digital conditioning 158, decimator/interpolator 160, and ambient subtractor 162.

Analog conditioning 152 may perform any suitable analog conditioning of the detector signal. The conditioning performed may include any type of filtering (e.g., low pass, high pass, band pass, notch, or any other suitable filtering), amplifying, performing an operation on the received signal (e.g., taking a derivative, averaging), performing any other suitable signal conditioning (e.g., converting a current signal to a voltage signal), or any combination thereof.

The conditioned analog signal may be processed by analog-to-digital converter 154, which may convert the conditioned analog signal into a digital signal. Analog-to-digital converter 154 may operate under the control of control circuitry 110. Analog-to-digital converter 154 may use timing control signals from control circuitry 110 to determine when to sample the analog signal. Analog-to-digital converter 154 may be any suitable type of analog-to-digital converter of sufficient resolution to enable a physiological monitor to accurately determine physiological parameters.

Demultiplexer 156 may operate on the analog or digital form of the detector signal to separate out different components of the signal. For example, detector current waveform 214 of FIG. 2B includes a red component corresponding to peak 226, an IR component corresponding to peak 230, and at least one ambient component corresponding to valley 230. Demultiplexer 156 may operate on detector current waveform 214 of FIG. 2B to generate a red signal, an IR signal, a first ambient signal (e.g., corresponding to the ambient component corresponding to valley 230 that occurs immediately after the peak 226), and a second ambient signal (e.g., corresponding to the ambient component corresponding to valley 230 that occurs immediately after the IR component 230). Demultiplexer 156 may operate under the control of control circuitry 110. For example, demultiplexer 156 may use timing control signals from control circuitry 110 to identify and separate out the different components of the detector signal.

Digital conditioning 158 may perform any suitable digital conditioning of the detector signal. Digital conditioning 158 may include any type of digital filtering of the signal (e.g., low pass, high pass, band pass, notch, or any other suitable filtering), amplifying, performing an operation on the signal, performing any other suitable digital conditioning, or any combination thereof.

Decimator/interpolator 160 may decrease the number of samples in the digital detector signal. For example, decimator/interpolator 160 may decrease the number of samples by removing samples from the detector signal or replacing samples with a smaller number of samples. The decimation or interpolation operation may include or be followed by filtering to smooth the output signal.

Ambient subtractor 162 may operate on the digital signal. In some embodiments, ambient subtractor 162 may remove dark or ambient contributions to the received signal.

The components of front end processing circuitry 150 are merely illustrative and any suitable components and combinations of components may be used to perform the front end processing operations.

The front end processing circuitry 150 may be configured to take advantage of the full dynamic range of analog-to-digital converter 154. This may be achieved by applying gain to the detection signal, or in embodiments of the present disclosure, applying gain and/or offset to the difference signal, by analog conditioning 152 to map the expected range of the signal to the full or close to full output range of analog-to-digital converter 154. The output value of analog-to-digital converter 154, as a function of the total analog gain applied to the detection signal (or difference signal), may be given as:

ADC Value=Total Analog Gain×[Ambient Light+LED Light]

Ideally, when ambient light is zero and when the light source is off, the analog-to-digital converter 154 will read just above the minimum input value. When the light source is on, the total analog signal provided to analog-to-digital converter 154 may be modified such that the output of analog-to-digital converter 154 is close to the full scale without saturating. Modifications of the signal may include subtracting an estimate signal, applying an offset, and applying a gain. These modifications may allow the full or a substantial amount of the dynamic range of analog-to-digital converter 154 to be used for representing the detection signal, thereby increasing the resolution of the converted signal. In some embodiments, where the input to the analog-to-digital to converter maps to a relatively smaller number of analog-to-digital conversion bits, the output of analog-to-digital converter 154 may include fewer bits of resolution. In some embodiments, the total analog gain and other modifications may be adjusted by such that small changes in the light level incident on the detector do not cause saturation of analog-to-digital converter 154. In some embodiments, passive or active filtering or signal modification techniques may be employed to reduce the contribution of a noise or other undesirable signal component from the input to analog-to-digital converter 154, thereby increasing the effective resolution of the digitized signal.

Back end processing circuitry 170 may include processor 172 and memory 174. Processor 172 may be adapted to execute software, which may include an operating system and one or more applications, as part of performing the functions described herein. Processor 172 may receive and further sensor signals received from front end processing circuitry 150. For example, processor 172 may determine one or more physiological parameters based on the received physiological signals. Processor 172 may include an assembly of analog or digital electronic components. Processor 172 may calculate physiological information. For example, processor 172 may compute one or more of blood oxygen saturation (e.g., arterial, venous, or both), pulse rate, respiration rate, respiration effort, blood pressure, hemoglobin concentration (e.g., oxygenated, deoxygenated, and/or total), any other suitable physiological parameters, or any combination thereof. Processor 172 may perform any suitable signal processing of a signal, such as any suitable band-pass filtering, adaptive filtering, closed-loop filtering, any other suitable filtering, and/or any combination thereof. Processor 172 may also receive input signals from additional sources not shown. For example, processor 172 may receive an input signal containing information about treatments provided to the subject from User Interface 180. Additional input signals may be used by processor 172 in any of the calculations or operations it performs in accordance with back end processing circuitry 170 or monitor 104.

Memory 174 may include any suitable computer-readable media capable of storing information that can be interpreted by processor 172. In some embodiments, memory 174 may store calculated values such as estimate signals, pulse rate, blood pressure, blood oxygen saturation, fiducial point locations or characteristics, an initialization parameter, any other calculated values, or any combination thereof. Calculated values may be stored in a memory device for later retrieval. This information may be data or may take the form of computer-executable instructions, such as software applications, that cause the microprocessor to perform certain functions and/or computer-implemented methods. Depending on the embodiment, such computer-readable media may include computer storage media and communication media. Computer storage media may include volatile and non-volatile, removable and non-removable media implemented in any method or technology for storage of information such as computer-readable instructions, data structures, program modules or other data. Computer storage media may include, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by components of the system. Back end processing circuitry 170 may be communicatively coupled with user interface 180 and communication interface 190.

User interface 180 may include user input 182, display 184, and speaker 186. User interface 180 may include, for example, any suitable device such as one or more medical devices (e.g., a medical monitor that displays various physiological parameters, a medical alarm, or any other suitable medical device that either displays physiological parameters or uses the output of back end processing 170 as an input), one or more display devices (e.g., monitor, personal digital assistant (PDA), mobile phone, tablet computer, any other suitable display device, or any combination thereof), one or more audio devices, one or more memory devices (e.g., hard disk drive, flash memory, RAM, optical disk, any other suitable memory device, or any combination thereof), one or more printing devices, any other suitable output device, or any combination thereof.

User input 182 may include any type of user input device such as a keyboard, a mouse, a touch screen, buttons, switches, a microphone, a joy stick, a touch pad, or any other suitable input device. The inputs received by user input 182 can include information about the subject, such as age, weight, height, diagnosis, medications, treatments, and so forth.

In an embodiment, the subject may be a medical patient and display 184 may exhibit a list of values which may generally apply to the patient, such as, for example, age ranges or medication families, which the user may select using user input 182. Additionally, display 184 may display, for example, an estimate of a subject's blood oxygen saturation generated by monitor 104 (referred to as an “SpO₂” measurement), pulse rate information, respiration rate and/or effort information, blood pressure information, hemoglobin concentration information, any other parameters, and any combination thereof. Display 184 may include any type of display such as a cathode ray tube display, a flat panel display such a liquid crystal display or plasma display, or any other suitable display device. Speaker 186 within user interface 180 may provide an audible sound that may be used in various embodiments, such as for example, sounding an audible alarm in the event that a patient's physiological parameters are not within a predefined normal range.

Communication interface 190 may enable monitor 104 to exchange information with external devices. Communications interface 190 may include any suitable hardware, software, or both, which may allow monitor 104 to communicate with electronic circuitry, a device, a network, a server or other workstations, a display, or any combination thereof. Communications interface 190 may include one or more receivers, transmitters, transceivers, antennas, plug-in connectors, ports, communications buses, communications protocols, device identification protocols, any other suitable hardware or software, or any combination thereof. Communications interface 190 may be configured to allow wired communication (e.g., using USB, RS-232, Ethernet, or other standards), wireless communication (e.g., using WiFi, IR, WiMax, BLUETOOTH, USB, or other standards), or both. For example, communications interface 190 may be configured using a universal serial bus (USB) protocol (e.g., USB 2.0, USB 3.0), and may be configured to couple to other devices (e.g., remote memory devices storing templates) using a four-pin USB standard Type-A connector (e.g., plug and/or socket) and cable. In some embodiments, communications interface 190 may include an internal bus such as, for example, one or more slots for insertion of expansion cards.

It will be understood that the components of physiological monitoring system 100 that are shown and described as separate components are shown and described as such for illustrative purposes only. In some embodiments the functionality of some of the components may be combined in a single component. For example, the functionality of front end processing circuitry 150 and back end processing circuitry 170 may be combined in a single processor system. Additionally, in some embodiments the functionality of some of the components of monitor 104 shown and described herein may be divided over multiple components. For example, some or all of the functionality of control circuitry 110 may be performed in front end processing circuitry 150, in back end processing circuitry 170, or both. In other embodiments, the functionality of one or more of the components may be performed in a different order or may not be required. In an embodiment, all of the components of physiological monitoring system 100 can be realized in processor circuitry.

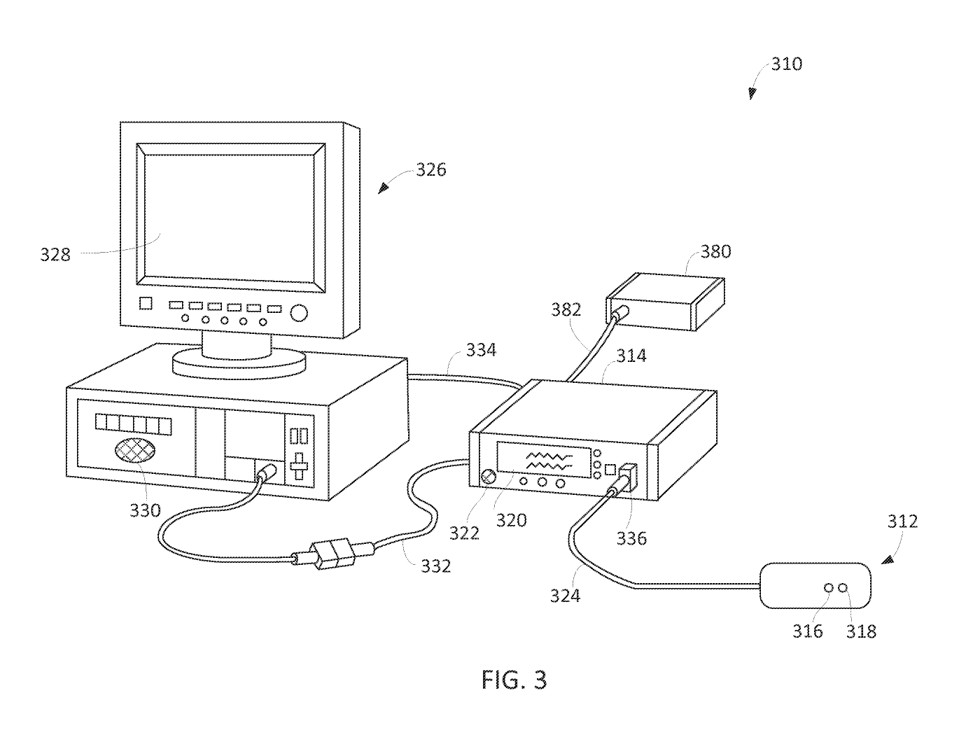

FIG. 3 is a perspective view of an illustrative physiological monitoring system 310 in accordance with some embodiments of the present disclosure. In some embodiments, one or more components of physiological monitoring system 310 may include one or more components of physiological monitoring system 100 of FIG. 1. Physiological monitoring system 310 may include sensor unit 312 and monitor 314. In some embodiments, sensor unit 312 may be part of an oximeter. Sensor unit 312 may include one or more light source 316 for emitting light at one or more wavelengths into a subject's tissue. One or more detector 318 may also be provided in sensor unit 312 for detecting the light that is reflected by or has traveled through the subject's tissue. Any suitable configuration of light source 316 and detector 318 may be used. In an embodiment, sensor unit 312 may include multiple light sources and detectors, which may be spaced apart. Physiological monitoring system 310 may also include one or more additional sensor units (not shown) that may, for example, take the form of any of the embodiments described herein with reference to sensor unit 312. An additional sensor unit may be the same type of sensor unit as sensor unit 312, or a different sensor unit type than sensor unit 312 (e.g., a photoacoustic sensor). Multiple sensor units may be capable of being positioned at two different locations on a subject's body.

In some embodiments, sensor unit 312 may be connected to monitor 314 as shown. Sensor unit 312 may be powered by an internal power source, e.g., a battery (not shown). Sensor unit 312 may draw power from monitor 314. In another embodiment, the sensor may be wirelessly connected (not shown) to monitor 314. Monitor 314 may be configured to calculate physiological parameters based at least in part on data relating to light emission and acoustic detection received from one or more sensor units such as sensor unit 312. For example, monitor 314 may be configured to determine pulse rate, respiration rate, respiration effort, blood pressure, blood oxygen saturation (e.g., arterial, venous, or both), hemoglobin concentration (e.g., oxygenated, deoxygenated, and/or total), any other suitable physiological parameters, or any combination thereof. In some embodiments, calculations may be performed on the sensor units or an intermediate device and the result of the calculations may be passed to monitor 314. Further, monitor 314 may include display 320 configured to display the physiological parameters or other information about the system. In the embodiment shown, monitor 314 may also include a speaker 322 to provide an audible sound that may be used in various other embodiments, such as for example, sounding an audible alarm in the event that a subject's physiological parameters are not within a predefined normal range. In some embodiments, physiological monitoring system 310 may include a stand-alone monitor in communication with the monitor 314 via a cable or a wireless network link. In some embodiments, monitor 314 may be implemented as display 184 of FIG. 1.

In some embodiments, sensor unit 312 may be communicatively coupled to monitor 314 via a cable 324 at port 336. Cable 324 may include electronic conductors (e.g., wires for transmitting electronic signals from detector 318), optical fibers (e.g., multi-mode or single-mode fibers for transmitting emitted light from light source 316), any other suitable components, any suitable insulation or sheathing, or any combination thereof. In some embodiments, a wireless transmission device (not shown) or the like may be used instead of or in addition to cable 324. Monitor 314 may include a sensor interface configured to receive physiological signals from sensor unit 312, provide signals and power to sensor unit 312, or otherwise communicate with sensor unit 312. The sensor interface may include any suitable hardware, software, or both, which may be allow communication between monitor 314 and sensor unit 312.

In some embodiments, physiological monitoring system 310 may include calibration device 380. Calibration device 380, which may be powered by monitor 314, a battery, or by a conventional power source such as a wall outlet, may include any suitable calibration device. Calibration device 380 may be communicatively coupled to monitor 314 via communicative coupling 382, and/or may communicate wirelessly (not shown). In some embodiments, calibration device 380 is completely integrated within monitor 314. In some embodiments, calibration device 380 may include a manual input device (not shown) used by an operator to manually input reference signal measurements obtained from some other source (e.g., an external invasive or non-invasive physiological measurement system).

In the illustrated embodiment, physiological monitoring system 310 includes a multi-parameter physiological monitor 326. The monitor 326 may include a cathode ray tube display, a flat panel display (as shown) such as a liquid crystal display (LCD) or a plasma display, or may include any other type of monitor now known or later developed. Multi-parameter physiological monitor 326 may be configured to calculate physiological parameters and to provide a display 328 for information from monitor 314 and from other medical monitoring devices or systems (not shown). For example, multi-parameter physiological monitor 326 may be configured to display an estimate of a subject's blood oxygen saturation and hemoglobin concentration generated by monitor 314. Multi-parameter physiological monitor 326 may include a speaker 330.

Monitor 314 may be communicatively coupled to multi-parameter physiological monitor 326 via a cable 332 or 334 that is coupled to a sensor input port or a digital communications port, respectively and/or may communicate wirelessly (not shown). In addition, monitor 314 and/or multi-parameter physiological monitor 326 may be coupled to a network to enable the sharing of information with servers or other workstations (not shown). Monitor 314 may be powered by a battery (not shown) or by a conventional power source such as a wall outlet.

In some embodiments, any of the processing components and/or circuits, or portions thereof, of FIGS. 1 and 3, including sensors 102, 312 and monitors 104, 314, 326, may be referred to collectively as processing equipment. For example, processing equipment may be configured to amplify, filter, sample and digitize an input signal from sensor 102 or 312 (e.g., using an analog-to-digital converter), and calculate physiological information from the digitized signal. Processing equipment may be configured to determine estimate signals, generate light drive signals, amplify, filter, sample and digitize detector signals, sample and digitize other analog signals, calculate physiological information from the digitized signal, perform any other suitable processing, or any combination thereof. In some embodiments, all or some of the components of the processing equipment may be referred to as a processing module.

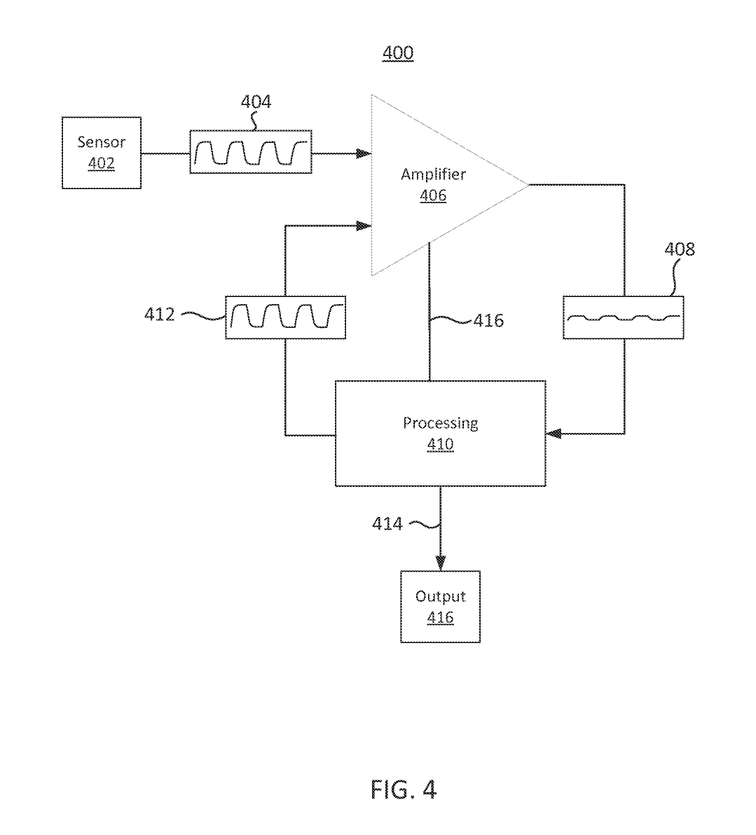

FIG. 4 is a block diagram of illustrative system 400 including an estimate signal in accordance with some embodiments of the present disclosure. System 400 includes sensor module 402, amplifier module 406, processing module 410, and output module 416. It will be understood that elements of system 400 may include any suitable elements, or be a part, of system 100 of FIG. 1 or system 310 of FIG. 3. For example, system 400 may be a part of front end processing 150 of FIG. 1. System 400 may be a part of monitor 314 of FIG. 3 and/or multi-parameter physiological monitor 326 of FIG. 3. In some embodiments, some or all components of system 400 and/or system 100 of FIG. 1 may use a common power supply. For example, both amplifier module 406 and processing module 410 may use a common supply voltage level. In some embodiments, using a shared voltage may reduce power consumption and avoid the use of boost circuitry and/or voltage conversion. In some embodiments, the use of lower voltage and shared supply rails (e.g., lower voltage power supplies) may allow lower power consumption, a smaller integrated circuit board area allocated to power supply, lower cost, and remove or reduce the use of boost, buck, and other power regulation circuitry.

FIG. 4 includes sensor signal 404, estimate signal 412, and difference signal 408. It will be understood that the particular signals shown in FIG. 4 are merely exemplary and that any suitable signal may be used. For example, signals may include any suitable amplitude modulations, frequency modulations, phase modulations, peak shapes, offsets, noise any other suitable features, or any combination thereof. It will also be understood that the signals shown in FIG. 4 may not be illustrated to scale. It will also be understood that the signals may include regular or non-regular elements such as those associated with physiological parameters that are not shown.

In some embodiments, the difference signal may include non-constant physiological information included in the sensor signal. In some embodiments, the difference signal may include information that corresponds to levels of SpO₂, respiration rates, respiration effort, pulse rhythm abnormalities such as arrhythmia, any other suitable variations, or any combination thereof. For example, the difference signal may include a modulation that corresponds to blood volume changes. The modulation may be used to determine physiological parameters.

Sensor module 402 may include some or all of sensor 102 of FIG. 1. For example, sensor module 402 may include one or more photodetector that converts a light signal received from a patient to an electrical signal. The output of sensor 402 may include sensor signal 404. For example, in an optical pulse oximeter, sensor signal 404 may be a photodetector current, such as detector current waveform 214 of FIG. 2B, representing the received attenuated light signal. Sensor module 402 may include any suitable filters, amplifiers, signal processing equipment, any other suitable equipment, or any combination thereof. In some embodiments, a substantially unprocessed sensor signal 404 is passed to amplifier module 406.

Amplifier module 406 may receive sensor signal 404 and estimate signal 412 (described below). Amplifier module 406 may generate difference signal 408 by determining a difference between the input signals. For example, estimate signal 412 may be subtracted from sensor signal 404 to generate difference signal 408. In some embodiments, amplifier module 406 may include a differential amplifier, a transimpedance amplifier, a differential transimpedance amplifier, summing amplifier, buffers, any other suitable amplifier or other elements configured to generate difference signal 408, or any combination thereof. In some embodiments, amplifier module 406 may be implemented using analog electronic components such as op-amps, inductors, capacitors, resistors, transistors, diodes, integrated circuits, current-to-voltage converters, voltage-to-current converters, any other suitable elements, and any combination thereof. Amplifier module 406 may receive signals with information corresponding to current levels, voltage levels, power levels, any other suitable metric, or any combination thereof. Amplifier module 406 may receive analog signals, digital signals, any other suitable signal type, or any combination thereof. For example, amplifier module 406 may receive a digital control signal to be used in amplifying analog signals. A transimpedance amplifier, as used herein, is an amplifier that converts a current input to a voltage output. A differential amplifier, as used herein, is an amplifier that determines the difference between two inputs. In an example, a dual-input, single output differential amplifier may be used to subtract estimate signal 412 from sensor signal 404. The common mode of the two signals may be substantially rejected, resulting in output differential signal 408 corresponding to the difference between the signals.

In some embodiments, amplifier module 406 may include one or more filters of any suitable configuration, for example, adaptive filters. Filters at the amplifier module, and at any other point, may receive information from an external source that is used to determine filtering parameters. For example, an electrosurgical unit may provide a synchronization line to the amplifier module that is used to remove noise associated with the electrosurgical unit.

Amplifier module 406 may include one or more amplifiers of any suitable gain. Gain, as used herein, refers to a comparison of the input signal level to the output signal level. For example, an amplifier that receives an input signal level of 1 volt and generates an output signal level of 1 volt has a gain of 1. An input of 1 volt and an output of 5 volts corresponds to a gain of 5. An offset, as used herein, refers to a positive or negative shift of a signal. For example, applying a +5V offset to a signal that ranges from 0V to 10V results in a signal that ranges from 5V to 15V. In some embodiments of amplifiers, such as transimpedance circuits, gain is controlled based on a feedback circuit between the output and the input. In some embodiments, amplifier module 406 may include one or more adjustable gain and offset settings. For example, gain that is controlled by a feedback resistor of an op-amp circuit may be adjusted by using an adjustable resistor. The gain-adjusting resistor may be a digitally controlled variable resistor (e.g., a digital potentiometer), a multi-tap resistor, switched resistors, a switching configuration that selectively includes and excludes components, any other suitable configuration, or any combination thereof. Gain may be fixed, controlled intermittently, controlled continuously, controlled during an initialization and/or calibration stage, controlled based on adjustments to the signal estimate, controlled based on adjustments to a light drive signal, controlled based on signal levels of differential signal 408, controlled using any other suitable technique, or any combination thereof. For example, system 400 may use a default gain of 1 at startup and may adjust the gain using a digitally controlled potentiometer such that the difference signal 408 is maintained at a particular signal level. In some embodiments, the gain level and offset may be controlled by and/or provided to processing module 410. Offsets may be implemented, for example, by using an analog adding circuit that is coupled to a DAC output. In an example, a 3V DC output from processing module 410 may be provided to an adder circuit (e.g., an op-amp summing amplifier) in amplifier module 406, where the 3V DC offset is added to the amplified or pre-amplified signal. In some embodiments, connection 416 provides information such as amplification and gain settings from processing module 410 to amplifier module 406.

Processing module 410 receives difference signal 408. Processing module 410 may include any suitable processing equipment, such as one or more processors, one or more micro-controllers, DACs, ADCs, any other suitable equipment, or any combination thereof. Processing module 410 generates, based in part on difference signal 408, estimate signal 412 and output signal 414. In some embodiments, estimate signal 412 corresponds to an estimate of sensor signal 404. In some embodiments, estimate signal 412 may be generated such that difference signal 408 is minimized.

Estimate signal 412 may include an estimate of non-physiological contributions and constant physiological contributions to the sensor signal. Non-physiological contributions may include, for example, changes in the level of light that relate to the turning on and off of light sources, ambient light signals, dark signal contributions, noise contributions, any other suitable contributions, or any combination thereof. Constant physiological contributions may include, for example, contributions that relate to skin pigmentation and tissue reflectivity. Constant physiological contributions may relate to the subject, but may remain relatively constant with respect to the physiological parameter under analysis (e.g., blood volume). It will be understood that when the light sources are on, a portion of the received light signal corresponds to varying physiological information, while a portion of the received light corresponds to non-physiological and constant physiological information. The non-physiological and constant physiological information may be relatively constant with respect to the time scale of the physiological variations, and thus may be included in the estimate signal. That is to say, the estimate signal may include a relatively predicable baseline associated with each portion of the light drive signal, where the baseline includes non-physiological and constant physiological contributions.

Estimate signal 412 may be based on prior sensor signals, prior difference signals, system information, system settings, user input, any other suitable information, or any combination thereof.

In some embodiments, estimate signal 412 may additionally include physiological contributions to the sensor signal, such as modulations in the signal amplitude that correspond to pulse, respiration, motion, any other physiological information, or any combination thereof. These contributions may be determined based on prior determination of physiological conditions, patient information, patient history, any other suitable information, or any combination thereof.

In some embodiments, minimizing difference signal 408 may allow the gain and/or offset of amplifier module 406 to be increased without saturating the input of processing module 410. For example, the input of processing module 410 may include an analog-to-digital converter (ADC). The gain and/or offset of amplifier module 406 may be adjusted such that the input to the ADC utilizes substantially the full scale input of the ADC, or any other desired amount. In some embodiments, the offset may be applied to difference signal 408 and/or the estimate signal 412 may be generated to cause a desired offset in difference signal 408.

Estimate signal 412 may be generated by processing module 410 using a DAC. The output may be a current controlled signal, a voltage controlled signal, a voltage signal including circuitry such as resistors to generate a current signal, any other suitable signal, or any combination thereof.

In some embodiments, estimate signal 412 initially is a zero amplitude waveform, and a small amount of gain is applied by amplifier module 406. The estimate may be iteratively refined based on the difference signal 408 received by processing module 410. The iterative refinement of both the gain and the signal estimate amplitudes may continue until difference signal 408 is minimized, until the difference signal 408 is below a threshold, until any other suitable point, or any combination thereof. In an example, the gain and/or signal estimate amplitudes may be initialized at a low level and increased until the difference signal is maintained at a particular level. In another example, the gain and/or signal estimate amplitudes may be initialized at a high level that is expected to saturate the ADC, and may be decreased until the difference signal is maintained at a particular level. In another example, the system may perform a binary search for the correct gain level and/or signal estimate levels by starting at an intermediate gain level and successively doubling or halving the amount of gain until the difference signal is maintained at a particular level. It will be understood that any suitable technique or combination of techniques may be used to determine gain level and signal estimate levels. It will be understood that gain may be adjusted separately and/or in combination with signal estimate levels and light drive levels.

Processing module 410 may update estimate signal 412 any at suitable rate and/or interval. For example, the estimate may be updated at the same rate that the ADC samples difference signal 408. In some embodiments, processing module 410 may include a sleep and/or low-power mode, in which received signals are stored in memory but not processed. In some embodiments, processing module 410 may generate the estimate at a DAC using a first direct memory access channel if a processing core of processing module 410 is in a sleep mode, and may receive ADC samples using a second direct memory access channel. Initial estimate signals may also include a predetermined estimate, an estimate based on system parameters, an estimate based on patient parameters, any other suitable estimate, or any combination thereof. Estimates may be averaged, for example using ensemble averaging, finite impulse response filtering, infinite impulse response filtering, adaptive filtering, any other suitable averaging or filtering, or any combination thereof. In an example, a received waveform (e.g., a sensor signal waveform) may be added into the estimate to produce an estimate in the next iteration. Repeated iterations may reduce the amplitude of the difference signal, allowing gain and/or LED brightness to be increased. In another example, estimate signal 412 may be updated when difference signal 408 is above a certain threshold. For example, estimate signal 412 may be updated when the absolute value or sum of the squared difference signal 408 is above a predetermined or adjustable threshold.

In some embodiments, a time delay may be used in generating the estimate signal. A time delay may correspond to a time delay of one or more analog components that process the difference signal. For example, the analog components of amplifier module 406 may introduce a time delay between the input to amplifier module 406 and the output of amplifier module 406. Processing module 410 may include a time delay in generate estimate signal 412 such that it is expected to be substantially synchronized with sensor signal 404. It will be understood that a time delay may include any suitable advancement or delays in the signal. Further, adjustments may be linear, non-linear, based on any other suitable technique, or any combination thereof. In some embodiments, a time delay may include a group delay, that is, a delay corresponding to the propagation of signal envelopes through components.

Processing module 410 may generate output signal 414 based in part on difference signal 408. Processing module 414 may combine information from difference signal 408 and a prior estimate signal 412 to generate output signal 414. Output signal 414 may be provided to output module 416 for further processing, such as for calculation of physiological parameters based on the output signal, for communication to one or more other devices, for display, for any other suitable purpose, or any combination thereof. Output module 416 may include any suitable display, further processing equipment, telemetry equipment, back end processing, user interface, communication interface, any other suitable information, or any combination thereof.

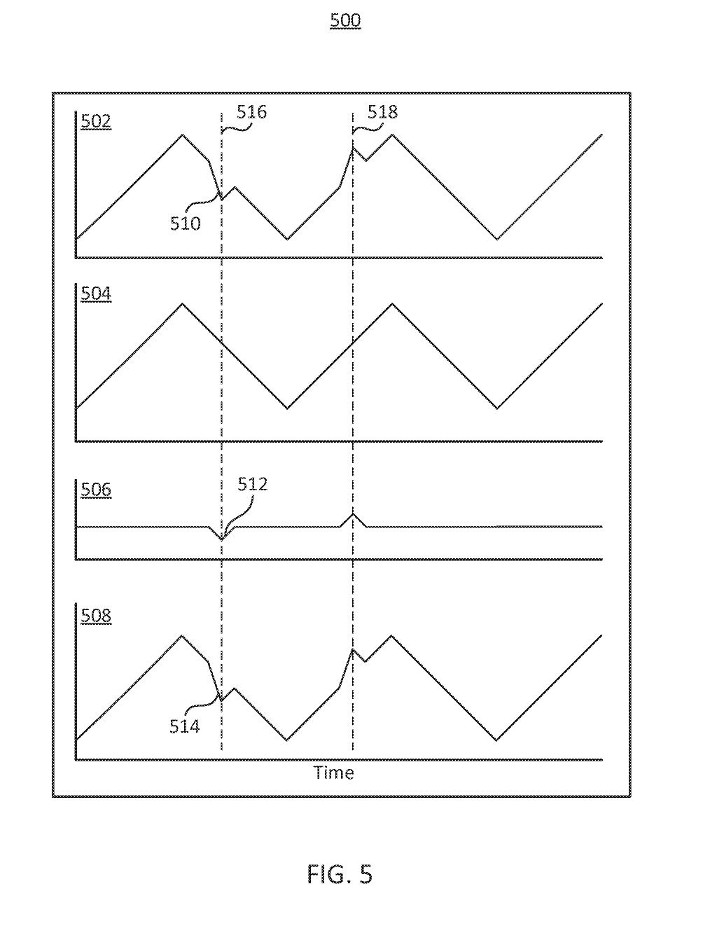

FIG. 5 shows illustrative signal plot window 500 in accordance with some embodiments of the present disclosure. It will be understood that the particular triangle wave signal illustrated in plot window 500 is merely exemplary and is used to illustrate some of the functionality of system 400. The particular features of the signals in plot 500 do not necessarily correspond to any physiological characteristics or phenomena, but rather generally illustrate the generation of a difference signal by subtraction of a signal estimate from a sensor signal. It will be understood that the system may use any suitable signals or combination of signals. It will be understood that in other examples, physiological information may correspond to changing peak heights in the triangle wave, other amplitude, frequency, and phase modulations, any other suitable changes in the signal, or any combination thereof. In the illustrated embodiment, the large triangular wave may correspond to non-physiological modulations of a received sensor signal, while the small peaks at time points 516 and 518 correspond to physiological information. By subtracting the non-physiological information from the sensor signal, a difference signal is generated including only the physiological information. In some embodiments, a larger gain may be applied to this difference signal than otherwise could be applied to the sensor signal without saturating the input of an ADC to which the amplified signal is provided.

In an example, sensor plot 502 corresponds to an output from sensor module 402 of FIG. 4, estimate plot 504 corresponds to an output from processing module 410 of FIG. 4, difference plot 506 corresponds to an output from amplifier module 406 of FIG. 4, and output plot 508 corresponds to an input to output module 416 of FIG. 4. It will be understood that the relative scale and amplitude of peaks 510, 512, and 514 may be exaggerated for clarity. The plots of plot window 500 are drawn with a common horizontal axis corresponding to time and vertical axes corresponding to signal amplitude. Signal amplitude may correspond to voltage, current, power, digital signal level, any other suitable metric, or any combination thereof. The signals may include analog and/or digital information.

Sensor plot 502 shows an illustrative sensor signal. In an example, the signal of sensor plot 502 may be an analog electrical current signal received from a photodetector of an optical pulse oximeter. In some embodiments, the sensor signal may represent a light signal that has been partially attenuated though interactions with a subject. Sensor plot 502 includes peak 510 in addition to an underlying sawtooth wave.

Estimate plot 504 shows an illustrative estimate of the sensor signal. In an example, the signal of estimate plot 504 may be an analog output of processing module 410 of FIG. 4. The estimate may include, for example, estimated values of the sensor signal.

Difference plot 506 shows an illustrative difference signal. In an example, the signal of difference plot 506 may be an analog output signal from amplifier module 406 of FIG. 4. In some embodiments, the signal of difference plot 506 is generated by subtracting the signal of estimate plot 504 from the signal of sensor plot 502. Peak 512 may correspond to peak 510 of sensor plot 502, as indicated by the vertical dotted line at time point 516. Thus, it will be understood that difference plot 506 corresponds to differences between the estimate signal and the output signal, as described above with regards to FIG. 4.

Output plot 508 shows an illustrative output signal. In some embodiments, output plot 508 shows a digitized signal. In an example, the signal of output plot 508 may correspond to an output provided to output module 416 of FIG. 4. In some embodiments, the signal of output plot 508 is generated by combining the signal of difference plot 506 with the signal of estimate plot 504. Peak 514 may correspond to peak 510 of sensor plot 502. It will be understood that while sensor plot 502 and output plot 508 appear similar, output plot 508 may correspond to an amplified and/or digitized signal that includes information corresponding to both the sensor signal and the estimate signal. In some embodiments, system 400 of FIG. 4 may increase the resolution and other quality metrics of the signal of output plot 508, for example as compared to a system that directly digitizes and outputs sensor plot 502. In an example, amplifier module 406 of FIG. 4 may generate an amplified difference signal that is digitized. The difference signal may be smaller than the sensor signal, allowing for a higher gain to be applied by amplifier module 406 of FIG. 4 to the difference signal without saturating the input of an analog-to-digital converter. The digital representation of the difference signal may then be combined with the estimate signal to reconstruct a high resolution digital representation of the sensor signal, shown in output plot 508.

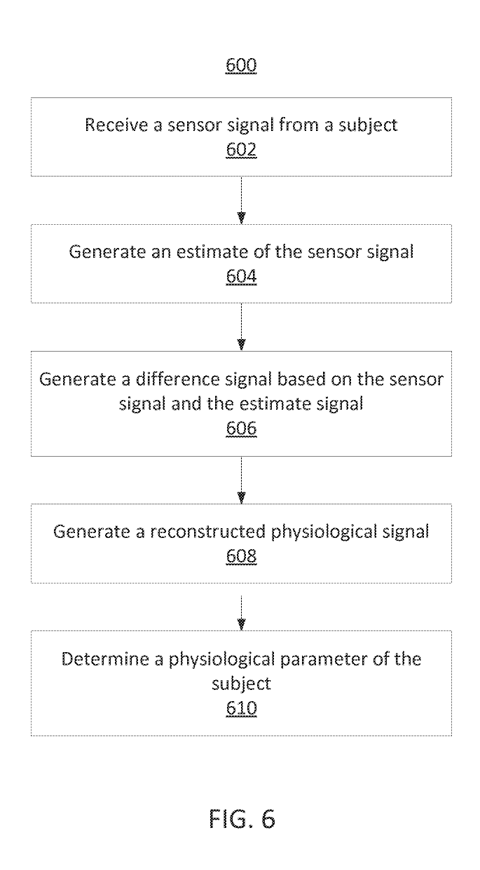

FIG. 6 shows illustrative flow diagram 600 including steps for determining a physiological parameter using an estimate of a sensor signal in accordance with some embodiments of the present disclosure.

In step 602, a sensor signal from a subject is received. The sensor signal may correspond to a signal received by detector 140 of FIG. 1, detector current waveform 214 of FIG. 2B, sensor signal 404 of FIG. 4, the signal of sensor plot 502 of FIG. 5, any other suitable signal, or any combination thereof. Sensor signals may correspond to sensor signals from a photodetector of a pulse oximeter, another optical sensor, an electrical sensor, a mechanical sensor, any other suitable sensor of any other suitable device capable of generating a sensor signal, or any combination thereof. In some embodiments, the sensor signal is an analog signal that includes physiological information such as blood volume.

In some embodiments, such as a pulse oximeter, the received signal may include a cycle of segments. For example, the repeating segments illustrated in FIG. 2B may be segments that repeat in a cycle. The cycle may include a first repeating segment that corresponds to an off period in a light drive modulation, a second repeating segment that corresponds to a first wavelength of light emitted by a pulse oximeter, and a second repeating segment corresponding to a second wavelength of light emitted by a pulse oximeter. It will be understood that the system may use any suitable number of segments in any suitable sequence. In some embodiments, a pulse oximeter may determine one or more amplitudes corresponding to the detected light in each repeating segment, and may use this information to determine physiological parameters.

In step 604, an estimate of the sensor signal is generated. The estimate of the sensor signal may correspond to, for example, estimate signal 412 of FIG. 4 and the signal of estimate plot 504 of FIG. 5. The estimate of the sensor signal may be based on prior received sensor signals, sensor probe information, system design parameters, user input, any other suitable information, or any combination thereof. The estimate may be used by the system to allow a higher resolution digitization of a desired portion of information that is contained within the sensor signal. For example, the estimate may be subtracted from the sensor signal and a gain may be applied to the difference signal. Because the difference signal may be relatively small, a relatively high gain may be applied.

In some embodiments, the estimate may represent the expected signal levels of both the non-physiological and constant physiological contributions to the sensor signal received in step 602. For example, the non-physiological contributions may include ambient light levels, LED light modulation, any other non-physiological contributions, or any combination thereof. Constant physiological contributions may include relatively constant contributions to the level of detected light in the sensor signal such as non-blood volume related tissue interactions, skin pigmentation, reflections of light attributable to bone and other structures, any other suitable relatively constant contributions, or any combination thereof. Physiological contributions may include changes in the amplitude of the sensor signal that correspond to changes in blood volume, changes in path length, patient motion, any other suitable physiological contributions, or any combination thereof. Changes in blood volume may correspond to cardiac pulses, respiration, any other suitable physiological changes, or any combination thereof.

In some embodiments, physiological information of the subject may be used in generating the estimate signal. Physiological information may include pulse rate, respiration rate, medical history, any other suitable information, or any combination thereof.]

In some embodiments, sensor signals may include generally repeating content. For example, modulation in the sensor signal may significantly correspond to a light drive modulation such as that shown in FIG. 2A. In some embodiments, characteristics of the sensor signal may change relatively slowly, that is to say, the peak heights, shapes, periodicity, and other characteristics remain largely the same between subsequent peaks and within a relatively short time period. Accordingly, information from prior received sensor signals may be used in generating an estimate signal. In some embodiments, the estimate signal may account for substantially all of the newly received sensor signal information except for physiological modulations. In an example, the portion of a received sensor signal during a particular on period that exceeds the ambient level may be 95% attributable to the light emission and 5% attributable to physiological modulations. That is, sensor signals may include repeating peaks, such as those shown in detector waveform 214 of FIG. 2B, that repeat with regular amplitude and frequency. The height of these peaks may vary over time, for example through a cardiac pulse cycle. The modulation may account for, for example, 1%-10% of the peak height. The signal estimate may include the 90% of light intensity during that repeating segment that does not include the varying physiological information. In some embodiments, the estimate may include estimates for each repeating segment. It will be understood that the above range of modulations is merely exemplary. For example, modulations on the order of 0.1% may occur in low perfusion patients. In another example, modulations in the 10%-30% may be processed.

In some embodiments, the estimate may be generated using a digital-to-analog converter that is a part of, for example, processing module 410 of FIG. 4 or any other suitable micro-controller. Components used to generate the estimate may be analog, digital, or any combination thereof. Any suitable components may be partially or fully integrated into a micro-controller. For example, an ADC and DAC may be integrated into a micro-controller that includes memory elements (e.g., direct memory access channels) for storing and writing signals from and to the converters, respectively. The estimate may include estimates of pulses due to system parameters such as light source switching, gain and/or offset adjustments, light source brightness adjustment, probe information, any other suitable system parameter, or any combination thereof. Estimates may additionally or alternatively include physiological parameters such as baseline shifts associated with respiration, pulses associated with cardiac pulses, any other suitable physiological information, or any combination thereof. In some embodiments, estimates may be generated based on an external information source such as a pulse rate provided by an EKG or a filter parameter provided by an external electrosurgical unit to block noise associated with that unit. In some embodiments, the estimate may take into account a time delay such as a group delay to account for, for example, propagation delays in analog amplification components. In embodiments where the sensor signal corresponds to a cycle of segments, the estimate may include a corresponding cycle of segments.

In step 606, a difference signal is generated based on the received sensor signal of step 602 and the estimate of step 604. In some embodiments, the difference signal includes physiological information that is included in the sensor signal. In some embodiments, the difference signal is generated by subtracting the estimate signal from the sensor signal. The difference signal may correspond to difference signal 408 of FIG. 4 and signal of difference plot 506 of FIG. 5.

In some embodiments, generating the difference signal includes inputting the sensor signal of step 602 and the estimate of step 604 into an amplifier. The output of the amplifier may correspond to the difference signal. The amplifier may correspond to amplifier module 406 of FIG. 4. For example, the amplifier may include a transimpedance amplifier, a differential amplifier, any other suitable amplifier, or any combination thereof. It will be understood that the difference signal may be generated using analog components, digital components, or a combination of analog and digital components. The difference signal may be generated by subtracting current levels, subtracting voltage levels, subtracting power levels, subtracting any other suitable metric levels, or any combination thereof. For example, a transimpedance amplifier may be used to subtract a current level from a DAC-generated estimate signal from the sensor signal and provide an amplified voltage output. Power requirements for the amplifier may be reduced, and the gain may be increased, in amplifying the difference signal rather than the sensor signal because the dynamic range of the difference signal is much smaller. Amplifying the difference signal also effectively adds the DAC resolution used to generate the signal estimate to the ADC resolution used to digitize the amplified difference signal. This may allow the system to use lower resolution and lower cost components while maintaining the same quality of determining physiological parameters.

In some embodiments, the difference signal includes primarily varying physiological information. It may be desirable to apply a high gain to the varying physiological information without amplifying non-physiological and constant physiological information. By applying a gain to the difference signal, the system may primarily amplify varying physiological information. This amplified signal may be digitized using, for example, an analog-to-digital converter (ADC). An amount of gain and an offset applied by an amplifier in generating the difference signal may be adjusted based on the ADC, such that the utilization of the dynamic range of the ADC input is optimized. In some embodiments, a large gain may be applied so that small current changes in the sensor signal may produce measureable voltage changes at the ADC. In some embodiments, the amount of gain and/or offset may be adjusted based on the utilization of the dynamic range of the ADC is maintained at a particular level. For example, too small of a gain may result in unused ADC input dynamic range, while too large of a gain may result in the amplifier output reaching the power supply maximum output level (e.g., the supply rail) and the output may be clipped. Too large of an applied gain may also result in saturating the ADC input.

In some embodiments, the gain and/or offset may be initialized at particular values, and may be increased or decreased such that the signal input to the ADC utilizes a particular fraction (e.g., 75%) of the ADC input's full scale. Offsets may be adjusted to center or otherwise align the signal with the range of the input. For example, gain may be increased when a relatively small amount of the ADC input range is being utilized, and gain may be reduced when the signal exceeds the range (e.g., signal clipping). In another example, an offset may be increase when the dynamic range of the signal is of a suitable amount, but it is not aligned with the input (e.g., a +2V offset may be applied to a signal that ranges from −2V to 8V when the ADC input range is 0V to 10V).

In some embodiments, the gain and offset applied by the amplifier may be adjusted by processing equipment in communication with the ADC. In some embodiments, the light drive signal provided to an LED such as light source 130 of FIG. 1 is adjusted alternatively or in addition to adjusting an amount of gain and/or offset in order to adjust the utilization of the ADC's input range. In some embodiments, gain and/or offset may be reduced if the signal is highly variable, and may be increased (and accordingly the portion of the input range used increased) when the signal is relatively regular.

In step 608, a digitized representation of the sensor signal may be reconstructed using the digitized difference signal and the estimate signal. For example, a digital estimate signal that corresponds to the signal used in generating the difference signal may be combined digitally with the digitized difference signal to generate a reconstructed signal. For example, as illustrated in output plot 508 of FIG. 8, an output signal may be generated as a digital signal. In some embodiments, the estimate signal that was provided to the amplifier module when the particular difference signal was generated may be used in reconstructing the sensor signal. Reconstructing may include adding the difference signal to the estimate signal. Reconstruction may include accounting for gains, offsets, time delays, other adjustments, or any combination thereof, that have been applied to component signals. For example, the difference signal and/or the estimate signal may be rescaled such that they may be added together digitally to generate a reconstructed signal. In some embodiments, generating the reconstructed signal may provide increased resolution of small variations in the sensor signal by allowing a high gain to be applied to the difference signal. In some embodiments, the resolution of the output signal may combine the resolution of the ADC used to digitize the difference signal with the resolution of the signal estimate. For example, where the difference signal is digitized using a 16 bit ADC and the estimate signal is generated using a 12 bit DAC, the resolution of the two converters is combined in reconstructing the digitized representation of the sensor signal.

In some embodiments, the reconstructed signal is used to generate a plethysmographic signal. Generating a plethysmography signal may include demultiplexing a time division multiplexed signal. In some embodiments, segments of the signal may include ambient signal levels and detected signal levels, for example as described for detector current waveform 214 of FIG. 2B. In some embodiments, multiple corresponding segments of a signal are combined using ensemble averaging in generating a plethysmographic signal and/or in determining information from a plethysmographic signal.

In step 610, a physiological parameter of the subject is determined. The physiological parameter may be determined based on the difference signal of step 606. For example, the physiological parameter may be determined based on the reconstructed representation of the sensor signal generated in step 608. Physiological parameters determined by the system may include oxygen saturation, blood pressure, heart rate, respiratory rate, respiratory effort, any other suitable parameter, or any combination thereof. Parameters may be determined using any suitable hardware or software techniques. In some embodiments, the determined physiological parameter may be provided for further processing, may be provided to a user using any suitable audio and/or video technique, may be communicated using any suitable communication technique, may be used in any other suitable processing, or any combination thereof.

It will be understood that the aforementioned steps of flow diagram 600 are exemplary and that in some implementations, steps may be added, removed, omitted, repeated, reordered, modified in any other suitable way, or any combination thereof. For example, in some embodiments, step 608 may not be performed and the physiological parameter may be determined in step 610 based on the difference signal generated in step 606.

The foregoing is merely illustrative of the principles of this disclosure and various modifications may be made by those skilled in the art without departing from the scope of this disclosure. The above described embodiments are presented for purposes of illustration and not of limitation. The present disclosure also can take many forms other than those explicitly described herein. Accordingly, it is emphasized that this disclosure is not limited to the explicitly disclosed methods, systems, and apparatuses, but is intended to include variations to and modifications thereof, which are within the spirit of the following claims.

What is claimed:

1. A method for processing a sensor signal in a physiological monitor, the method comprising:
receiving, using processing equipment, an analog sensor signal corresponding to an intensity of detected light attenuated by a subject, wherein the analog sensor signal comprises at least a first segment corresponding to an ambient light interval and a second segment correspond to an emitted light interval;
generating, using the processing equipment, an analog estimate signal, wherein the analog estimate signal comprises an estimate corresponding to at least a portion of the first segment and an estimate corresponding to at least a portion of the second segment;
generating, using the processing equipment, an analog difference signal based at least in part on the analog sensor signal and the analog estimate signal; and
determining, using the processing equipment, a physiological parameter of the subject based at least in part on the analog difference signal.

2. The method of claim 1, wherein the analog estimate signal comprises an estimate of non-physiological contributions and constant physiological contributions to the analog sensor signal.

3. The method of claim 1, wherein the analog estimate signal comprises an estimate based at least in part on the analog sensor signal.

4. The method of claim 1, wherein the analog sensor signal comprises a pulse oximeter sensor signal comprising a repeating cycle of segments, wherein the repeating cycle comprises:
a first repeating segment corresponding to the first segment where no light is being emitted by the pulse oximeter sensor;
a second repeating segment corresponding to the second segment where a first wavelength of light is being emitted by the pulse oximeter sensor;
a third repeating segment correspond to an emitted light interval where a second wavelength of light is being emitted by the pulse oximeter sensor; and
wherein the analog estimate signal is generated to cancel a substantial portion of the non-physiological contributions to the analog sensor signal during the first repeating segment and non-physiological and constant physiological contributions during the second and third repeating segments.

5. The method of claim 1, wherein generating the analog difference signal comprises inputting the analog sensor signal and the analog estimate signal into an amplifier and outputting the analog difference signal from the amplifier.

6. The method of claim 5, wherein the amplifier comprises an amplifier selected from the group consisting of a transimpedance amplifier, a differential amplifier, and any combination thereof.

7. The method of claim 1, wherein generating the analog difference signal comprises subtracting the analog estimate signal from the analog sensor signal.

8. The method of claim 1, further comprising generating a reconstructed physiological signal based at least in part on the estimate of the sensor signal and the analog difference signal, and wherein the physiological parameter is determined from the reconstructed physiological signal.

9. The method of claim 1, wherein the physiological parameter is selected from the group consisting of oxygen saturation, pulse rate, respiration rate, respiration effort, blood pressure, hemoglobin concentration, and any combination thereof.

10. The method of claim 1, wherein the estimate of the sensor signal is generated based at least in part on a time delay of one or more analog components that process the difference signal.

11. The method of claim 1, further comprising applying a gain to the difference signal, wherein the gain is adjusted based on the input range of an analog-to-digital converter.

12. A system for processing a sensor signal in a physiological monitor, the system comprising:
processing equipment configured to perform operations comprising:
receiving an analog sensor signal corresponding to an intensity of detected light attenuated by a subject, wherein the analog sensor signal comprises at least a first segment corresponding to an ambient light interval and a second segment correspond to an emitted light interval;
generating an analog estimate signal, wherein the analog estimate signal comprises an estimate corresponding to at least a portion of the first segment and an estimate corresponding to at least a portion of the second segment;
generating an analog difference signal based at least in part on the analog sensor signal and the analog estimate signal; and
determining a physiological parameter of the subject based at least in part on the analog difference signal.

13. The system of claim 12, wherein the analog estimate signal comprises an estimate of non-physiological contributions and constant physiological contributions to the analog sensor signal.

14. The system of claim 12, wherein the analog estimate signal comprises an estimate based at least in part on the analog sensor signal.

15. The system of claim 12, wherein the analog sensor signal comprises a pulse oximeter sensor signal comprising a repeating cycle of segments, wherein the repeating cycle comprises:
a first repeating segment corresponding to the first segment where no light is being emitted by the pulse oximeter sensor;
a second repeating segment corresponding to the second segment where a first wavelength of light is being emitted by the pulse oximeter sensor;
a third repeating segment correspond to an emitted light interval where a second wavelength of light is being emitted by the pulse oximeter sensor; and
wherein the analog estimate signal is generated to cancel a substantial portion of the non-physiological contributions to the analog sensor signal during the first repeating segment and non-physiological and constant physiological contributions during the second and third repeating segments.

16. The system of claim 12, wherein generating the analog difference signal comprises inputting the analog sensor signal and the analog estimate signal into an amplifier and outputting the analog difference signal from the amplifier.

17. The system of claim 16, wherein the amplifier comprises an amplifier selected from the group consisting of a transimpedance amplifier, a differential amplifier, and any combination thereof.

18. The system of claim 12, wherein generating the analog difference signal comprises subtracting the analog estimate signal from the analog sensor signal.

19. The system of claim 12, wherein the processing equipment is configured to perform operations further comprising generating a reconstructed physiological signal based at least in part on the estimate of the sensor signal and the difference signal, and wherein the physiological parameter is determined from the reconstructed physiological signal.

20. The system of claim 12, wherein the physiological parameter is selected from the group consisting of oxygen saturation, pulse rate, respiration rate, respiration effort, blood pressure, hemoglobin concentration, and any combination thereof.

21. The system of claim 12, wherein the estimate of the sensor signal is generated based at least in part on a time delay of one or more analog components that process the difference signal.

22. A method for determining a physiological parameter of a subject, comprising:
receiving, using processing equipment, a signal representing intensity of light attenuated by a subject;
generating, using the processing equipment, an estimate signal based at least in part on an idealized light intensity signal;
generating, using the processing equipment, a difference signal representing a difference between the received signal and the estimate signal;
amplifying, using the processing equipment, the difference signal; and
determining, using the processing equipment, a physiological parameter of the subject based at least in part on the amplified difference signal.

23. The method of claim 22, wherein determining the physiological parameter comprises generating a reconstructed signal based on the amplified difference signal and a portion of the estimate signal.

24. The method of claim 22, wherein the estimate signal includes repeating portions corresponding to repeating portions of the received signal, and wherein the difference signal includes modulations of the received signal caused by a physiological process of the subject.
